(19) United States
(12) Patent Application Publication
(10) Pub. No.: US 2014/0276516 A1
(43) Pub. Date: Sep. 18, 2014

(54) ABSORBENT ARTICLE HAVING IMPROVED PERMEABILITY-DEPENDENT ABSORPTION UNDER PRESSURE

(71) Applicant: INDEVCO LTD., Beirut (LB)

(72) Inventors: Georges Dagher, Beirut (LB); Rana Khalil, Beirut (LB); Rabih Osta, Beirut (LB); Chadi Mattar, Beirut (LB)

(73) Assignee: INDEVCO Ltd., Beirut (LB)

(21) Appl. No.: 13/828,568

(22) Filed: Mar. 14, 2013

(51) Int. Cl. A61L 15/20; A61L 15/26; A61L 15/24

(52) U.S. Cl. CPC A61L 15/20; A61L 15/24; A61L 15/26
USPC 604/372; 604/375; 604/377

(57) ABSTRACT

An absorbent article, including a liquid permeable top sheet, a pulpless absorbent core, the core including synthetic fibers and a super absorbent material, the super absorbent material having a PDAUP greater than 10 g/g, and a liquid impermeable backsheet, wherein the absorbent core is positioned between the top sheet & the backsheet.

### BACKGROUND

Mass production of disposable diapers began in the 1960s. The early diaper cores consisted of 100% fluff pulp. The performance of these early cores was not sufficiently efficient, as the liquid absorbed by the fluff pulp was easily expressed from the structure upon application of pressure to a saturated core. The introduction of super absorbent polymers (SAP) in the early 1980s allowed for higher performance diaper cores. The use of SAP allowed for the reduction or complete removal of the bulky fluff pulp from the absorbent core. This allowed the diapers to become thinner while maintaining an improved performance. The reduction in physical size was a desired feature for consumers and also led to savings in handling & transportation of such products.

Absorbent products such as baby diapers, feminine hygiene pads and adult incontinence products are typically constructed of several different types of materials. These products typically include a permeable non-woven top sheet, an impermeable back sheet and an absorbent core sandwiched therebetween. The absorbent core typically consists of either wood fluff or synthetic fiber and a liquid-absorbing polymer, e.g. a SAP.

The super absorbent polymers are typically based on acrylic acid and sodium acrylate, and are able to swell so as to absorb and retain a quantity of liquid several times the weight of the SAP, consequently forming a gel. The gel formation from the absorbed liquid allows the SAP to act as a fluid locking system, even under pressure.

Many factors can influence the performance of the super absorbent polymer, including the form of the SAP particles, and the positioning of the SAP in the absorbent core. An inadequate distribution of the SAP through the core (for example, having a majority of the SAP near the top of the core) can lead to a gel blocking problem, and, consequently, to leakage. In such cases, the SAP absorbs liquid and then swells to form a gel that fills the voids between the pulp fibers, therefore blocking the top layer to the admittance of additional fluid.

In general, some of the important performance attributes of an absorbent core of a diaper are functional capacity, rate of absorption, core stability in use, absorption under load, and the permeability-dependent absorption under pressure of the SAP.

The ability of a SAP to absorb liquid under pressure, as well as the ability of a swollen SAP to retain liquid under pressure, are important properties for any super absorbent polymer. The absorption under load (AUL) of a SAP is a measure of the capacity of the SAP to absorb a saline solution (for example an 0.9% NaCl solution) under a specified enclosing pressure.

After the SAP absorbs a liquid and forms a gel, the gel should resist the forcing out of the liquid by applied pressure. This ability is related to the gel strength of the SAP, which is a measure of the ability of the gel to support a known weight without losing liquid. This ability is also linked to the behavior of the SAP under pressure, called permeability-dependent absorption under pressure (PDAUP). The PDAUP is a measure of the capacity of a swollen SAP to absorb a saline solution (for example an 0.9% NaCl solution) under a specified enclosing pressure, as well as the permeability of the swollen SAP. The permeability of the SAP is important to characterize the distribution of liquid within a swollen SAP layer. The PDAUP is expressed as the amount, in grams, of an aqueous solution that a superabsorbent material can absorb per gram thereof, under a load of 0.7 pounds per square inch, in the time span of one hour.

### SUMMARY

According to at least one exemplary embodiment, an absorbent article is disclosed. The absorbent article can include a liquid permeable top sheet, a pulpless absorbent core, the core including synthetic fibers and a super absorbent material, the super absorbent material having a PDAUP greater than 10 g/g, and a liquid impermeable backsheet, wherein the absorbent core is positioned between the top sheet & the backsheet.

### BRIEF DESCRIPTION OF THE FIGURES

Advantages of embodiments of the present invention will be apparent from the following detailed description of the exemplary embodiments. The following detailed description should be considered in conjunction with the accompanying figures in which:

FIGS. 1a-1c are top views of exemplary embodiments of absorbent articles having an absorbent core.

FIG. 2 is a cross sectional view of an exemplary embodiment of an absorbent article.

FIGS. 3a-3c are cross-sectional views of exemplary embodiments of absorbent cores, illustrating positioning of the super absorbent polymer within the core.

FIGS. 4a-4c illustrate exemplary absorbent core wrap configurations.

FIG. 5 illustrates an exemplary embossed pattern for an absorbent synthetic core.

FIG. 6 is a diagram of an exemplary absorbent core forming unit for an absorbent article.

FIG. 7 illustrates an exemplary apparatus useful for carrying out the procedure for calculating permeability dependent absorption under pressure.

### DETAILED DESCRIPTION

Aspects of the invention are disclosed in the following description and related drawings directed to specific embodiments of the invention. Alternate embodiments may be devised without departing from the spirit or the scope of the invention. Additionally, well-known elements of exemplary embodiments of the invention will not be described in detail or will be omitted so as not to obscure the relevant details of the invention. Further, to facilitate an understanding of the description discussion of several terms used herein follows.

As used herein, the word “exemplary” means “serving as an example, instance or illustration.” The embodiments described herein are not limiting, but rather are exemplary only. It should be understood that the described embodiment are not necessarily to be construed as preferred or advantageous over other embodiments. Moreover, the terms “embodiments of the invention”, “embodiments” or “invention” do not require that all embodiments of the invention include the discussed feature, advantage or mode of operation.

Embodiments disclosed herein relate to absorbent products, such as disposable diapers, incontinence pads, sanitary napkins, and the like, which have an absorbent core that includes synthetic fibers and super absorbent polymers (SAP) having a permeability-dependent absorption under pressure (PDAUP) above 10 g/g at 0.7 psi. Such an absorbent material can simultaneously optimize absorption under load as well as permeability, thereby providing improved absorption, rewetting and comfort.

According to at least one exemplary embodiment, about 30% to about 100% percent of the SAP can be attached to the synthetic fibers.

According to at least one exemplary embodiment, the absorbent core can be made from about 50% to about 95% of super absorbent polymer having a 60-minute PDAUP>10 g/g. According to further exemplary embodiments, the absorbent core can be made from about 50% to about 95% of super absorbent polymer having a 60-minute PDAUP ranging from about 14 g/g to about 22 g/g. The superior permeability-dependent absorption under load and permeability properties of the superabsorbent material can improve liquid absorption and retention by the absorbent article and can prevent excessive rewetting and leakage.

According to at least one exemplary embodiment, an absorbent article can include an absorbent core having a super absorbent polymer and synthetic fibers. The absorbent article structure can result in a thin product. Such absorbent articles can include disposable diapers, incontinence pads, sanitary napkins, and the like, wherein the absorbent core includes synthetic fibers and super absorbent particulate having a 60 minute PDAUP>10 g/g. The SAP can be between about 50% to about 95% of the weight of the absorbent core.

FIGS. 1a-1c are plan views of exemplary embodiments of absorbent articles 100, for example a diaper or sanitary pad. The absorbent article 100 can include the absorbent core 102 and the chassis 104. The absorbent core may have any desired shape. For example, as shown in FIGS. 1a-1c, the absorbent core 102 may be rectangular, rounded-rectangular, or may have an anatomically-conforming shape; however the absorbent core 102 should not be construed as being limited to solely the illustrated shapes.

FIG. 2 is a cross sectional view of a typical absorbent article 100. The absorbent article 100 can include a permeable top sheet 106, a liquid impermeable back sheet 108 and the absorbent core 102 sandwiched therebetween. Additional layers, such as an acquisition layer 109, may be disposed between top sheet 106 and absorbent core 102 to facilitate faster liquid penetration into the core. The acquisition layer can be made of air-through bonded bi-component fibers or thermally bonded webs of polyester fibers. The absorbent core 102 can include a synthetic fiber matrix 110 positioned adjacent SAP 112, with both matrix 110 and SAP 112 enclosed by wrap 114. The SAP 112 and synthetic fiber matrix 110 can be coupled to the wrap 114 by any suitable adhesive 116. The absorbent core 102 may be disposed between back sheet 108 and top sheet 106. Furthermore, absorbent core 102 can be positioned such that the synthetic fiber matrix is disposed between the permeable top sheet 106 and the SAP 112.

In some exemplary embodiments, the SAP 112 and synthetic fiber matrix 110 can be contained within a wrap 114. Wrap 114 can be made of a material that can impede the passage of SAP 112 therethrough. Such materials can include tissue, for example a single-ply white tissue having a high wet strength, and synthetic non-woven materials, for example polyolefin fibers such as polyethylene or polypropylene fibers.

Top sheet 106 may be any permeable polymeric non-woven sheet known in the art. A suitable top sheet 106 may be made from, for example, perforated plastic films, polyolefin fibers (e.g., polyethylene or polypropylene fibers), or combinations thereof. It should be understood that additional layers may be present between absorbent core 102 and top sheet 106.

Back sheet 108 can be any impermeable polymeric plastic and/or non-woven sheet known in the state of the art. For example, a suitable back sheet may be made from films of polyethylene, polypropylene, polyester, nylon, polyvinyl chloride or blends of these materials. It should be understood that additional layers may be present between the absorbent core and the back sheet.

Absorbent core 102 can include SAP 112 in addition to a synthetic fiber matrix 110. The synthetic material used for matrix 110 can be, for example, polyolefins (e.g., polypropylene and polyethylene), rayon, polycarbonates, bicomponent fibers, cellulose acetate, and so forth. Such fibrous material is known in the art as tow, and may be a crimped tow. Tow is a continuous band composed of several thousand filaments, which can be held loosely together by a crimp, i.e., a wave configuration set into the band during its manufacture. The tow band can be formed by combining the filaments from several spinnerets. The combined bundle of filaments can then be crimped to facilitate tow band cohesion as well as suitable bulk and firmness when processed. Prior to making an absorbent composite that includes a tow fiber, the tow fiber can typically be unwound, opened, and then cut at various lengths, so as to provide a fibrous mass of material. Tow having crimped filaments can be easier to open.

The tow fiber can be any continuous or discontinuous thermoplastic filament tow fiber that is capable of being opened and used in combination with SAP 112 in an absorbent core 102. The denier per fiber (dpf) of the tow fiber can be in the range of about 1 dpf to about 9 dpf, for example about 5 dpf to about 8 dpf. For products having the same weight, filaments of lower dpf may provide increased surface area and increased moisture absorption. Total denier may vary within the range of about 26,000 denier to about 40,000 denier, depending upon the process used.

The SAP 112 are polymers that can absorb and retain large amounts of a liquid relative to their own mass. A suitable SAP may be, for example, a sodium-neutralized cross-linked polyacrylate. Such an SAP may be formed by polymerization of a large quantity of units of acrylic acid blended with sodium hydroxide in the presence of an initiator. The acrylic acid and sodium hydroxide may be mixed together in the presence of water, an initiator, and a cross-linker. The resulting solution can undergo a polymerization process to form a three-dimensional polymer chain network, which can take the form of an aqueous gel. Subsequently, the aqueous gel may be chopped, crushed and dried to form SAP granules. The SAP granules can then be ground and sieved to obtain a desired particle size. At this point, the SAP granules can be further cross-linked so as to obtain desired absorbance under pressure characteristics.

In some exemplary embodiments, the SAP 112 can have a 60-minute PDAUP value that is higher than 10 g/g. In further exemplary embodiments, the SAP 112 can have a 60-minute PDAUP value ranging from about 14 g/g to about 22 g/g.

FIGS. 3a-3c illustrate exemplary positioning of the SAP 112 within the core 102. As shown in FIG. 3a, the SAP 112 can be positioned such that, when core 102 is disposed within an absorbent article 100, the SAP 112 is proximate back sheet 108, while the synthetic fiber matrix is proximate top sheet 106. Both SAP 112 and matrix 110 can be coupled by an adhesive 116 to wrap 114. As shown in FIG. 3b, the SAP 112 can be enclosed in the synthetic fiber matrix 110. The matrix 110 can then be coupled by an adhesive 116 to wrap 114. Another exemplary position of SAP 112 within core 102 is shown in FIG. 3c, where, when core 102 is disposed within an absorbent article 100, the synthetic fiber matrix 110 is disposed proximate top sheet 106, a portion of SAP 112 is positioned proximate bottom sheet 108, and additional portions of SAP 112 are positioned proximate top sheet 106 on the sides of the synthetic fiber matrix 110 and proximate wrap 114.

FIGS. 4a-4c illustrate exemplary configurations of wrap 114. As shown in FIG. 4a, wrap 114 may be provided as a single sheet of wrap, having a portion overlapping itself and coupled to itself. As shown in FIG. 4b, wrap 114 may be provided as two sheets of wrap, a first sheet 114a enclosing the bottom and sides of the absorbent core, and a second sheet 114b enclosing the top of the absorbent core and having a portion thereof overlapping and coupled to first sheet 114a. As shown in FIG. 4c, wrap 114 may be provided as two sheets of wrap, a first sheet 114a enclosing the top and sides of the absorbent core, and a second sheet 114b enclosing the bottom and sides of the absorbent core, with the two sheets 114a, 114b being coupled to each other at the sides of the absorbent core.

In yet other exemplary embodiments, the SAP 112 may be attached to the synthetic fiber matrix 110, the wrap 114, or the back sheet 108. This can facilitate increasing the performance of the absorbent garment 100. Various chemical, mechanical, thermal or electrical means of attaching the SAP 112 to the synthetic matrix 110 can be employed. Any attachment means can be suitable as long as it does not interfere with the ability of the SAP 112 to absorb liquid. Such means include adhesives, heat sonication, embossing or sonic bonding patterns. It should also be understood that a combination of treatments can be used. FIG. 5 shows an exemplary embossed pattern 120 applied to a portion of an absorbent product. The embossed pattern 120 can subdivide the core 102 into pockets or cells containing SAP 112 and synthetic matrix 110. This can facilitate fixing the SAP 112 in place, thereby improving the performance of the absorbent article.

FIG. 6 illustrates an apparatus 200 for forming an exemplary absorbent article 100. The apparatus 200 can utilize any desired type of tow fiber. The apparatus 200 can include a tow feeder 202 that is capable of feeding the opened tow fiber 220 into a core forming station 204. A SAP feeder system 206 can provide SAP 222 to core forming station 204. The core forming station 204 can combine the tow fiber 220 and SAP 222 to form an absorbent composite core 224. The absorbent composite 224 can then be wrapped by a tissue or polyolefin layer 226, to which an adhesive can be applied by an applicator 208. Subsequently, the wrapped absorbent composite 228 can pass through an embossing station 210.

FIG. 7 illustrates a PDAUP testing apparatus 300. The permeability-dependent absorption under load test measures the ability of a swollen material to absorb saline solution under a specified pressure. The PDAUP is expressed as the amount, in grams, of an aqueous sodium chloride solution (0.9 weight percent NaCl) absorbed by 1 g of material, during one hour, and under a load of approximately 0.7 psi.

The PDAUP testing apparatus 300 can include a tray 302, a filter plate 304, a cylinder 306, and a piston weight 310.

Tray 302 may be a petri dish or similar vessel and may be sized to accommodate the apparatus and to supply sufficient saline solution to meet the absorption capacity of the sample for the duration of the test. Filter plate 304 can have a porosity of zero. Cylinder 306 can be made from polymethyl methacrylate cylinder, and can include a filter screen 308 at the bottom thereof. The filter screen 308 can be a 400 mesh nylon cloth filter screen, a stainless steel screen, or the like. The piston weight 310 can have sufficient size and mass so as to apply a pressure of 0.7 psi when used in combination with a piston 312. Piston 312 can be formed from polytetrafluoroethylene.

The aqueous NaCl solution may be prepared as follows: approximately 9 g of sodium chloride crystals are weighed and added along with 1000 mL of de-ionized water to a clean dry 1000 ml flask. The concentration of the saline solution is measured with a saltmeter to insure 0.9% concentration.

The test procedure may be as follows: samples for the PDAUP test are prepared by rotating a sample container of super absorbent polymer several times in order to obtain a homogeneous product. Subsequently, 5.0±0.1 g from the SAP sample are weighed & recorded as m₀. The SAP 314 is then evenly distributed onto the filter screen 308 of cylinder 306 where the piston 310 is inserted. The weight of the completed cylinder apparatus is recorded as m₁.

The filter plates 304 are inserted in the tray 302. Subsequently, the 0.9% aqueous NaCl solution is added until the surface of the liquid 316 reaches the same level as the surface of the filter plate 304. The completed cylinder apparatus is then placed on the filter plate 304 along with the weight 310.

After 60 minutes, the weight 310 is removed & the weight of the cylinder apparatus is recorded as m₂.

The PDAUP is then calculated & expressed in g/g:

$$PDAUP = \frac{{m}_{2} - {m}_{1}}{{m}_{0}} ,$$

where m₀ is the mass, in grams, of the dry SAP 314; m₁ is the mass, in grams, of cylinder 306, filter screen 308, and dry SAP 314; and m₂ is the mass, in grams, of cylinder 306, filter screen 308, and SAP 314 with absorbed solution 316.

The foregoing description and accompanying figures illustrate the principles, preferred embodiments and modes of operation of the invention. However, the invention should not be construed as being limited to the particular embodiments discussed above. Additional variations of the embodiments discussed above will be appreciated by those skilled in the art.

Therefore, the above-described embodiments should be regarded as illustrative rather than restrictive. Accordingly, it should be appreciated that variations to those embodiments can be made by those skilled in the art without departing from the scope of the invention as defined by the following claims.

1. An absorbent article, comprising:
a liquid permeable top sheet,
a pulpless absorbent core, the core including synthetic fibers and a super absorbent material, the super absorbent material having a PDAUP between about 14 g/g and about 22 g/g; and
a liquid impermeable backsheet;
wherein the absorbent core is positioned between the top sheet & the backsheet and
wherein PDAUP is a measurement of the capacity of a swollen super absorbent material to absorb a saline solution under an enclosing pressure and the permeability of the swollen super absorbent material, and is measured as the amount of a 0.9 weight percent NaCl solution absorbed by an amount of super absorbent material during one hour, under a load of approximately 0.7 pounds per square inch.

2. (canceled)

3. The absorbent article of claim 1, wherein the super absorbent material is a sodium neutralized, cross-linked polyacrylate.

4. The absorbent article of claim 1, wherein the absorbent core further comprises a wrap.

5. The absorbent article of claim 4, wherein the wrap is formed from one of a polyolefin, a tissue, and a synthetic non-woven material.

6. The absorbent article of claim 1, wherein the synthetic fibers are tow fibers selected from the group consisting of:
cellulose acetate fibers, polypropylene fibers, rayon fibers, polyacrylonitril fibers, polypropylene and polyethylene bicomponent fibers, cotton fibers and cotton linter fibers.

7. The absorbent article of claim 1, wherein the synthetic fibers are colored.

8. The absorbent article of claim 4, wherein:
the super absorbent polymer is coupled to one of the synthetic fibers and the wrap;
the super absorbent polymer is coupled by one or more of a chemical coupling and a mechanical coupling.

9. The absorbent article of claim 8, wherein the chemical coupling is an adhesive.

10. The absorbent article of claim 8, wherein the mechanical coupling is a heated embossing.

11. The absorbent article of claim 1, wherein the absorbent core further comprises an acquisition layer.

12. The absorbent article of claim 1, wherein the super absorbent material is about 50% to about 95% by weight of the absorbent core.

13. The absorbent article of claim 1, further comprising an embossed pattern applied to a portion of the absorbent article.
